(19) United States
(12) Patent Application Publication
(10) Pub. No.: US 2020/0384501 A1
(43) Pub. Date: Dec. 10, 2020

(54) THIN FILM COATING PROCESSES AND COMPOSITIONS

(71) Applicant: Benefit Coatings Inc., Stratford, CT (US)

(72) Inventors: Craig J. Berry, Stratford, CT (US); Adam Berry, Stratford, CT (US); Kim Berry, Stratford, CT (US)

(73) Assignee: Benefit Coatings Inc., Stratford, CT (US)

(21) Appl. No.: 16/875,166

(22) Filed: May 15, 2020

Related U.S. Application Data
(60) Provisional application No. 62/850,229, filed on May 20, 2019.
(60) Provisional application No. 62/849,260, filed on May 17, 2019.

(51) Int. Cl. B05D 7/00; B05D 7/24; A61K 9/70; B05C 21/00

(52) U.S. Cl. CPC B05D 7/544; B05D 7/24; B05C 21/00; B05D 7/584; A61K 9/703

(57) ABSTRACT

The present disclosure relates to thin film coating processes, compositions so formed and intermediates used and formed therein using multiple coating stations and multiple drying stations in the thin film coating processes.

### CROSS-REFERENCE TO RELATED APPLICATIONS

This application claims priority to and the benefit of U.S. Provisional Patent Application Ser. No. 62/849,260 filed May 17, 2019 and U.S. Provisional Patent Application Ser. No. 62/850,229 filed May 20, 2019, the disclosures of which are incorporated herein by reference in its entirety.

### FIELD

The aspects of the present disclosure relate to thin film, multi-layer films, coated non wovens and transdermal patch coating processes, compositions so formed and intermediates used and formed therein.

### BACKGROUND

Coated products, such as oral and vaginal mucosal dissolving thin films, transdermal patches, non-woven coated bandages and hemostats, non-dissolving films, and more are compositions that can be used to quickly and easily ingest, absorb, and treat, with healthy, therapeutic and nutritionally beneficial ingredients. Such coated products thereof may include the above beneficial ingredients as well as other additional ingredients that may not be compatible with one another in a single composition, but the inclusion of which may improve various properties of the coated compositions, and also may aid in the manufacture of these coated products.

It would be beneficial to have compositions and methods to create coated products that include multiple coated ingredients that have different chemical and organoleptic properties while each such ingredient maintaining its own chemical integrity.

### SUMMARY

In one embodiment, a method of making a thin film composition is provided. The method includes depositing a first coating on a substrate; drying the first coating to remove moisture or solvent therefrom; depositing a second coating onto the first coating; and drying the second coating to remove moisture or solvent therefrom.

In another embodiment, a method of making a thin film composition is provided. The method includes depositing a first coating on a substrate; drying the first coating to remove moisture or solvent therefrom; depositing a second coating onto the first coating; depositing a third coating onto the second coating; and drying the second coating and the third coating to remove moisture or solvent therefrom.

In another embodiment, a coating system making a thin film composition is provided. The coating system includes a supply of substrate material; a first coating station into which the substrate material is introduced and to which a first coating is deposited onto the substrate material; a first drying station after the first coating station into which the substrate material including the first coating leaving the first coating station is introduced; a second coating station after the first drying station into which the substrate material including the first coating leaving the first drying station is introduced and to which a second coating is deposited onto the first coating; and a second drying station after the second coating station into which the substrate material including the first coating and the second coating leaving the second coating station is introduced.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a schematic illustrative of an embodiment of the present disclosure;

FIG. 2 is a cross-section view of a thin layer composition embodiment of the present disclosure;

FIG. 3 is a schematic illustrative of another embodiment of the present disclosure; and

FIG. 4 is a cross-section view of a thin layer composition embodiment of the present disclosure;

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The aspects of the embodiments of the present disclosure include compositions as well as methods of making same as well as intermediates used therein including thin film compositions including ingestible and/or therapeutic thin film compositions, such as, for example, mucosal dissolving thin film, transdermal patch, bandage, hemostats, non-dissolving film and edible film compositions. More particularly, the aspects of the embodiments of the present disclosure include compositions as well as methods of making same as well as intermediates used therein including coated thin film compositions including coated ingestible and/or coated therapeutic thin film compositions, such as, for example, coated mucosal dissolving thin film, coated transdermal patch, coated bandage, coated non-dissolving film and coated edible film compositions.

More particularly, the aspects of the embodiments of the present disclosure include methods of making coated thin film compositions as well as the coated thin film and intermediate thin film compositions themselves including applying more than one coating to a substrate (e.g., a release liner) and subjecting the substrate to more than one drying step during the coating process, including, for example, a drying step after one or more coating steps.

Thin film compositions and methods can be improved by the embodiments of the present disclosure by the addition of materials after a preliminary coating or coatings and drying process(es) are applied but before a secondary in-line coating(s) and drying process(es). The aspects of the disclosed embodiments provide for the addition of materials, after a first or preliminary coating(s) process, but before a secondary drying process or processes are performed, in a single pass. The web or substrate is prepared to receive additional materials during an additional in-process coating or coatings, prior to the secondary drying process or processes.

Among other benefits, the aspects of the disclosed embodiments can provide significant time savings in the coating process of multi-layer film compositions.

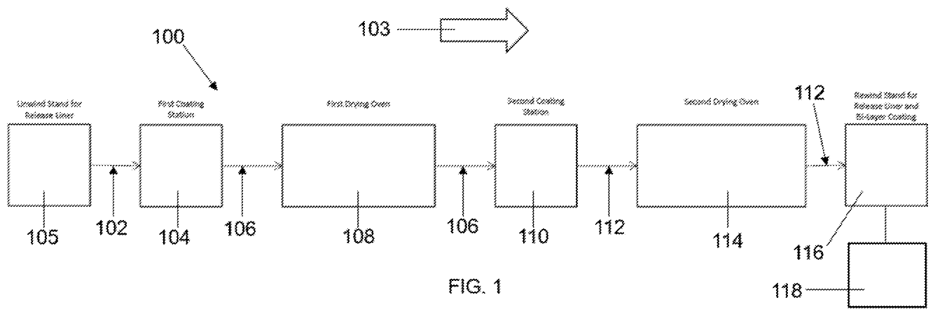

FIG. 1 schematically illustrates one embodiment of the present disclosure and includes a coating system or coating line 100. The coating system or coating line 100 includes a supply of substrate (e.g., a release liner) 102 on, for example, an unwind stand 105 and proceeds through the coating system or coating line 100 in the direction of arrow 103. The substrate material 102 may be fed through a first coating station 104 to form a substrate material with a first coating 106. After the first coating station 104, the substrate with a first coating 106 then passes through a first drying oven 108 (drying oven and drying station can be used interchangeably herein to mean the same component) at a temperature sufficient to heat the first coating and flash off moisture (e.g., water) or solvent to either fully or partially dry the coating material on substrate with a first coating 106 that was deposited thereon at the first coating station 104, including all, substantially all, most or a portion of the moisture.

After the first drying station 108, the substrate with a first coating 106 then passes through a second coating station 110 (which is different from the first coating station 104) that deposits a second coating onto the first coating. After the second coating station 110, the substrate with a first coating and second coating 112 then passes through a second drying oven 114 (which is different from the first drying oven station 108) at a temperature sufficient to heat the second coating and flash off moisture (e.g., water) or solvent in the second coating material on the substrate with a first coating and second coating 112 and may also flash off some moisture(e.g., water) or solvent that remains in the coating material that was deposited at the first coating station 104. After the second drying oven station 114, the substrate with a first coating and second coating 112 can be collected, for example, on a rewind stand 116. Movement of the substrate 102 and the coated versions thereof (substrate with a first coating 106 and substrate with a first coating and second coating 112) can be pulled through the coating system or coating line 100 using, for example, a drive system 118 (e.g., an electric motor) connected to the rewind stand 114 to pull the substrate 102 and the coated versions thereof through the coating system or coating line 100.

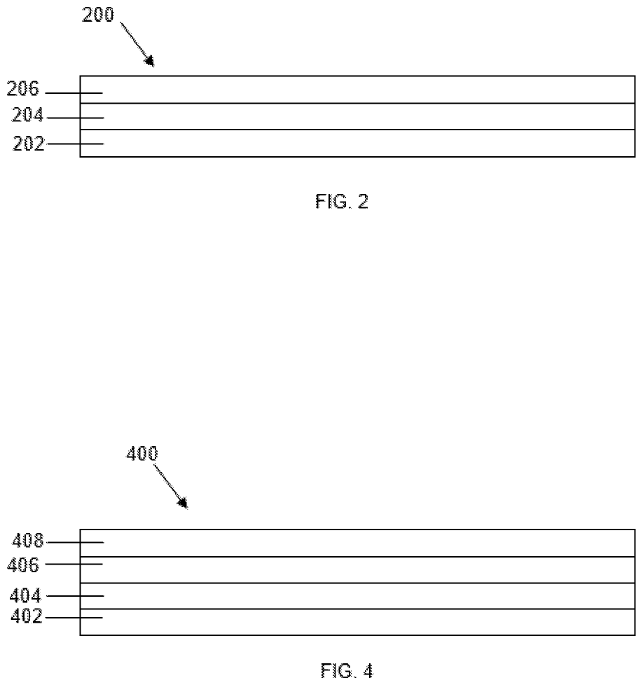

FIG. 2 illustrates a cross-sectional view of one embodiment of the substrate with a first coating and second coating 110 from FIG. 1. FIG. 2 includes coated substrate 200, substrate 202, first coating 204 deposited at the first coating station 104 of FIG. 1 and second coating 206 deposited at the second coating station 110 of FIG. 1.

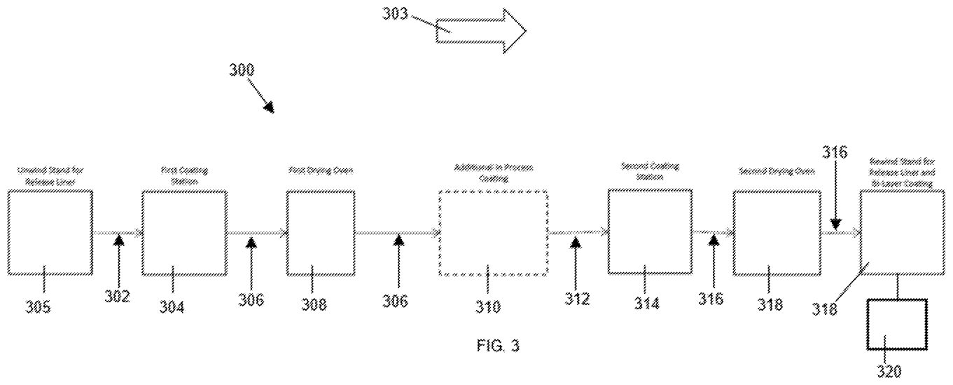

FIG. 3 schematically illustrates another embodiment of the present disclosure and includes a coating system or coating line 300. The coating system or coating line 300 includes a supply of substrate (e.g., a release liner) 302 on, for example, an unwind stand 305 and proceeds through the coating system or coating line in the direction of arrow 303. The substrate material 302 may be fed through a first coating station 304 to form a substrate material with a first coating 306. After the first coating station 304, the substrate with a first coating 306 then passes through a first drying oven 308 at a temperature sufficient to heat the first coating and flash off moisture (e.g., water) or solvent to either fully or partially dry the coating material on substrate with a first coating 306 that was deposited thereon at the first coating station 304, including all, substantially all, most or a portion of the moisture (e.g., water) or solvent.

After the first drying station 308, the substrate with a first coating 306 then passes through a second coating station 310 (which is different from the first coating station 104) that deposits a second coating onto the first coating. After the second coating station 310, the substrate with a first coating and second coating 312 then passes through a third coating station 314 (which is different from the first coating station 306 and the second coating station 310) that deposits a second coating onto the first coating. After the third coating station 314, the substrate with a first coating, second coating and third coating 316 then passes through a second drying oven 318 (which is different from the first drying oven station 308) at a temperature sufficient to heat the second and third coatings and flash off moisture (e.g., water) or solvent in the second and third coating material on the substrate with a first coating, second coating and third coating 316 and may also flash off some moisture (e.g., water) or solvent that remains in the coating material that was deposited at the first coating station 304. After the second drying oven station 318, the substrate with a first coating, second coating and third coating 316 is collected, for example, on a rewind stand 318. Movement of the substrate 302 and the coated versions thereof (substrate with a first coating 306, substrate with a first coating and second coating 310 and substrate with a first coating, second coating and third coating 316) can be pulled through the coating system or coating line 300 using, for example, a drive system 320 (e.g., an electric motor) connected to the rewind stand 318 to pull the substrate 302 and the coated versions thereof through the coating system or coating line 300.

FIG. 4 illustrates a cross-sectional view of one embodiment of the substrate with a first coating, second coating and third coating 314 from FIG. 3. FIG. 4 includes coated substrate 400, substrate 402, first coating 404 deposited at the first coating station 304 of FIG. 3, a second coating 406 deposited at the second coating station 310 of FIG. 3 and a third coating 408 deposited at the third coating station 314 of FIG. 3.

FIGS. 1 and 3 illustrate exemplary thin film coating processes (and system components) incorporating aspects of the disclosed embodiments. With reference to FIGS. 1 and 3, the first coating in the preliminary stage can be dried in an initial oven so that it exits the initial oven either fully or partially dried, as included in the embodiments of the present disclosure. This occurs prior to the application of material or materials by second coating, for example, in the FIG. 1 embodiment and the second and third coatings, for example, in the FIG. 3 embodiment.

Thin film compositions and coated thin film compositions of the present disclosure include embodiment of the present disclosure with a substrate (e.g., a release liner) as well as those that may be produced with a substrate (e.g., a release liner),but the substrate (e.g., a release liner) is removed before or after the coated substrates of the present disclosure are cut to a desired size and shape including substrate removing immediately prior to use by, for example, the purchaser or consumer of the product.

The substrate (e.g., a release liner) of embodiments of the present disclosure should be impermeable to the coating materials, in particular, the coating material deposited to be in contact with the substrate and allow the coating material to disperse evenly thereon. This also allows for ease of removal of the substrate from the coated compositions of the present embodiment. Examples of suitable substrates include films such as plastic or polyester films, polypropylene, polycarbonate, non-siliconized polyethylene terephthalate film, non-siliconized Kraft paper, polyethylene impregnated Kraft paper, metal belts, voltage or corona treated belts, drum dryers, silicone coated material (e.g., a silicone coated belt) and polytetrafluorethylene-impregnated glass fabric. Other examples of substrates can include a non-woven or woven fabric material, such as, for example, (e.g., cotton, hemp, rayon, polyester, cellulose acetate, polypropylene, polyethylene, polycarbonate, polyester, polyvinyl chloride). Multiple substrates may also be employed. The substrate may be coated with a release coating, such as, for example, silicone.

The coating materials of the embodiments of the present disclosure may include, for example, various polymers and optionally, various plasticizers and when applied the coating materials are in water or a suitable solvent. Acceptable polymers can include, for example, polysaccharide-based or polysaccharide and glycoprotein-based gums such a, for example, pullulan, locust bean gum, xanthan gum, guar gum, alginates (e.g., Alginic acid, also called algin, sodium alginate), gum Arabic, pectin, xanthan gum, gelatin, starches (including corn, potato, rice or tapioca), modified starches, matltodextrins, wheat gluten, carboxymethylcellulose, carrageenan konjac, locust bean gum or combinations thereof.

The coating materials of the embodiments of the present disclosure may include, for example, various therapeutically active or nutritionally beneficial agents including, for example, agents such as analgesics, decongestants, bronchodilators and other antiasthmatic agents, cardiovascular agents such as beta-blockers, ACE inhibitors, diuretics, antithrombics, etc., diabetic agents, antihistamines, anesthetics, antifungals, antinauseants, antiemetics, antibacterial agents, antifungal agents, corticosteroids, neurological agents, anti-inflammatories, vaccines, biological agents (vaccines, blood components, allergens, proteins, hormones, antibodies, etc. including products like Humira®, Enbrel® and Remicade®), wound healing agents and anticonvulsants. Vitamins (particularly A, C, D and E) can also be exemplary therapeutically active and nutritionally beneficial agents.

The second coating step can also include the addition of an agent that may chemically or physically modify the first coating layer. For example, a first coating may be cross linked by the addition of the second coating that includes a cross linking agent for the particular polymer of the first coating.

The application of each of the coating materials in embodiments of the present disclosure (including first, second and third coating stations included in the embodiments of FIGS. 1 and 3) can be through any suitable coating application process, including for example, but not limited to, slot die, spray coating, powder brushing, knife over roll, gravure, reverse roll, placement, etc. In some embodiments, the coating can be applied using a variable speed drive controlling the volume of application. The coating stations of the embodiments of the present disclosure can include an aqueous based mixture, solvent based mixture, dry powder, granule, or other wet or dry applications. This can include, for example, the use of a gear pump or air pressure if it was a spray or speed if it was a rotating brush. Any number of application methods may be used or implemented, depending on the material being applied. An exemplary implementation includes a coating line with a drive control (e.g., drive systems 114 and 320 of FIGS. 1 and 3, respectively) that can control the speed of the coating systems or coating lines and, thusly the dwell time of the substrates and coated substrates at the coating stations, drying ovens and space in between. A non-woven can be spray coated in this way before it goes into the second oven. The temperatures of the drying ovens or stations can also be separately controlled.

In some embodiments of the present disclosure, additional steps can occur in the second coating station and other coating stations followed by one or more subsequent drying ovens that are preceded by a preliminary stage including the first coating station. In some other embodiments of the present disclosure, additional steps occur after a preliminary stage of multiple coating stations and one or more drying stations at the beginning of the coating system or coating line and include the additional steps occurring in one or more subsequent coating stations followed by one or more drying stations station. The processes of the disclosed embodiments are a continuous motion. There is no interruption of the substrate or web proceeding from one station to the next station. In this manner, the speed and efficiency of the overall coating process is improved. In alternative embodiments, the system described herein can be configured to provide the additional steps at any suitable point of the overall process, not including the second coating station or subsequent coating stations. The aspects of the disclosed embodiments are directed to applying the additional process in a single pass and in a continuous motion.

In some embodiments, after the additional process or steps, the multilayered coating then enters a secondary oven, also referred to as the second drying station or subsequent drying stations before the coated substrate is collected, to complete the drying process.

In accordance with the aspects of embodiments of the present disclosure, the coating created in the preliminary stage (e.g., the first coating station and first drying oven of FIGS. 1 and 3) is modified during what is described herein as an additional in-process coating by the addition of materials that improve the coating or coated composition formed thereby in some way. The additional steps of the present disclosure can occur in embodiments of the present disclosure after the first drying oven, as exemplified in FIGS. 1 and 3 as well as a preliminary stage of other embodiments of the present disclosure. These additional in-process coatings or additions are included in coatings applied after the preliminary stage of the process and can include additional materials in such coatings or adjustments to the process steps, for example, but are not limited to the following.

Texture modification components. Changes to texture can modify dissolution or stickiness of the coated compositions so the coatings do not stick to the inner surface of a pouch/cassette, or each other when packaged). Examples of such additions include physically embossing the film or adding a hydrophobic starch.

Flavor or sensation modification components. Many actives or primary ingredients in edible films can have unpleasant tastes that can be improved by the addition of a second layer of flavor masking material, flavor, or encapsulations. Examples of sensation modifiers can include cooling agents such as peppermint, spearmint, wintergreen, menthol, etc. or warming agents such as cinnamon. Flavoring agents can also include, for example, cherry, chocolate, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durian, green tea, grapefruit, banana, etc. or combinations thereof.

Addition of temperature sensitive component where the drying process of coating and the temperature sensitive component therein need to conducted at a lower temperature, including a much lower temperature than the first drying or previous drying process(es) so as not to compromise, disrupt or denigrate the temperature sensitive actives. Examples of such additions can include components with a poor stability profile, such as, for example, menthol, biologics, probiotics, etc. Such a difference in temperature could be, for example, the first drying station is at a temperature above room temperature and the second drying station is at a temperature at or slightly above room temperature.

Addition of barrier layers. For example, a second or subsequent layer that can protect the first layer or earlier applied layers and change its or their dissolution profile(s) and act as a barrier to delay release of components of the first layer. Examples of such additions include membranes, such as, for example, ethylene vinyl acetate or occlusive barriers such as polyester, polyurethane, or polyethylene.

Addition of backing layers. For example, a second or subsequent layer that can protect the first layer or earlier applied layers from outside environments, e.g., air, water, moisture, etc.

Addition of multiple therapeutically active or nutritionally beneficial agents ingredients that are incompatible with one another such that they need to be included in different coating layers or the agent in one layer requires a different dissolution profile from the agent in another layer.

Addition of microneedle arrays such as solid microneedles, coated microneedles, hollow microneedles, dissolving microneedles, or other microneedle arrays.

Addition of biologics, such as vaccines (e.g., a COVID-19 vaccine, a rotavirus vaccine, a measles vaccine, etc.), blood components, allergens, proteins, hormones, antibodies, DNA sequences, etc. where the drying process of the coating including such the biologic need to conducted at a lower temperature, including a much lower temperature, than the first or previous drying process(es) so as not to compromise, disrupt or denigrate the temperature sensitive actives. Such a difference in temperature could be, for example, the first drying station is at a temperature above room temperature and the second drying station is at a temperature at or slightly above room temperature.

Addition of electronic or smart components, such as conductive components or enhancers, microchips, sensors, batteries, diagnostics devices, communication devices, etc.

All publications, including but not limited to, issued patents, patent applications, and journal articles, cited in this application are each herein incorporated by reference in their entirety.

Thus, while there have been shown, described and pointed out, fundamental novel features of the present disclosure as applied to the exemplary embodiments thereof, it will be understood that various omissions and substitutions and changes in the form and details of devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A method of making a thin film composition, comprising:
depositing a first coating on a substrate;
drying the first coating to remove moisture or solvent therefrom;
depositing a second coating onto the first coating; and
drying the second coating to remove moisture or solvent therefrom.

2. The method according to claim 1, wherein depositing the first coating occurs at a first coating station, drying the first coating occurs at a first drying station, depositing the second coating occurs at a second coating station and drying the second coating occurs at a second drying station.

3. The method according to claim 2, wherein the first coating station and the second coating station are different and the first drying station and the second drying station are different.

4. The method of claim 1, wherein the depositing a first coating on a substrate and drying the first coating to remove moisture therefrom is a preliminary stage in the method of making the thin film composition and the preliminary stage includes depositing at least one additional coating to the first coating prior to the drying the first coating and the drying step includes drying the first coating and the at least one additional coating to remove moisture therefrom.

5. The method of claim 1, wherein the step of drying the first coating and the step of drying the second coating occur at different temperatures.

6. The method of claim 1, wherein the second coating includes at least one of the following a texture modification component, a flavor modification component, a sensation modification component, a temperature sensitive component where the second drying step is at a lower temperature than the temperature of the first drying step, a microneedle array, an electronic or smart component.

7. The method of claim 6, wherein the temperature sensitive component is a biologic including a vaccine, a blood components, an allergen, a protein, a hormone, an antibody or a DNA sequence.

8. The method of claim 7, wherein the vaccine is a COVID-19 vaccine.

9. The method of claim 1, wherein the second coating includes a barrier layer to protect the first layer or change the first layer's dissolution profile or a backing layer to protect the first layer from an external environment.

10. A method of making a thin film composition, comprising:
depositing a first coating on a substrate;
drying the first coating to remove moisture or solvent therefrom;
depositing a second coating onto the first coating;
depositing a third coating onto the second coating; and
drying the second coating and the third coating to remove moisture or solvent therefrom.

11. The method according to claim 10, wherein depositing the first coating occurs at a first coating station, drying the first coating occurs at a first drying station, depositing the second coating occurs at a second coating station, depositing the second coating occurs at a second coating station and drying the second coating and the third coating occurs at a second drying station.

12. The method according to claim 2, wherein the first coating station, the second coating station and the third coating station are different and the first drying station and the second drying station are different.

13. The method of claim 1, wherein the step of drying the first coating and the step of drying the second coating and the third coating occur at different temperatures.

14. The method of claim 1, wherein at least one of the second coating and the third coating includes at least one of the following a texture modification component, a flavor modification component, a sensation modification component, a temperature sensitive component where the second drying step is at a lower temperature than the temperature of the first drying step, a microneedle array, an electronic or smart component.

15. The method of claim 6, wherein the temperature sensitive component is a biologic including a vaccine, a blood component, an allergen, a protein, a hormone, an antibody or a DNA sequence.

16. The method of claim 7, wherein the vaccine is a COVID-19 vaccine.

17. The method of claim 1, wherein at least one of the second coating and the third coating includes a barrier layer to protect the first layer or change the first layer's dissolution profile or a backing layer to protect the first layer from an external environment.

18. A coating system making a thin film composition, comprising:
a supply of substrate material;
a first coating station into which the substrate material is introduced and to which a first coating is deposited onto the substrate material;
a first drying station after the first coating station into which the substrate material including the first coating leaving the first coating station is introduced;
a second coating station after the first drying station into which the substrate material including the first coating leaving the first drying station is introduced and to which a second coating is deposited onto the first coating; and
a second drying station after the second coating station into which the substrate material including the first coating and the second coating leaving the second coating station is introduced.

19. The coating system according to claim 18, further including a collection apparatus after leaving the second coating station for receiving the substrate material including the first coating and the second coating.

20. The coating system according to claim 18, wherein the first drying station subjects the substrate material including the first coating to a first temperature, the second drying station subjects the substrate material including the first coating and the second coating to a second temperature and the first temperature is higher than the second temperature.
